(19) United States
(12) Patent Application Publication
(10) Pub. No.: US 2023/0152293 A1
(43) Pub. Date: May 18, 2023

(54) ANALYZING GENETIC MATERIAL OF MICROORGANISMS TO DETERMINE THE MOVEMENT OF CARBON-BASED GAS

(71) Applicants: Caroline Burke, Houston, TX (US); Jordan Sawadogo, Houston, TX (US); John Haggerty, San Diego, CA (US); Mathias Schlecht, Kingwood, TX (US); Thomas Ishoey, Encinitas, CA (US); Cooper Gill, Salado, TX (US)

(72) Inventors: Caroline Burke, Houston, TX (US); Jordan Sawadogo, Houston, TX (US); John Haggerty, San Diego, CA (US); Mathias Schlecht, Kingwood, TX (US); Thomas Ishoey, Encinitas, CA (US); Cooper Gill, Salado, TX (US)

(21) Appl. No.: 17/986,643

(22) Filed: Nov. 14, 2022

Related U.S. Application Data
(60) Provisional application No. 63/278,966, filed on Nov. 12, 2021.

(51) Int. Cl. G01N 33/24; C12Q 1/6888; C12Q 1/64; G01N 33/18

(52) U.S. Cl. CPC G01N 33/241; C12Q 1/64; C12Q 1/6888; G01N 33/1826

(57) ABSTRACT

Samples are collected from a first wellbore and a second wellbore. Genetic material is extracted from the samples and analyzed to determine microorganisms present in subsurface geological features through which the first wellbore and the second wellbore pass. Movement of microorganisms originating in subsurface geological features at the location of the first wellbore to subsurface geological features at the location of the second wellbore can indicate movement of a carbon-based gas between the first wellbore and the second wellbore.

### PRIORTY CLAIM AND CROSS-REFERENCE TO RELATED APPLICATION

This application claims priority to U.S. Provisional Pat. Application Serial No. 63/278,966, filed on Nov. 12, 2021, and entitled “TOTAL FLUID MONITORNG USING DNA DIAGNOSTICS”, which is incorporated by reference herein in its entirety.

### TECHNICAL FIELD

The present application relates generally to the technical field of monitoring the microbial profile of fluids derived from subsurface samples to determine movement of one or more gases within one or more subsurface geological features.

### BACKGROUND

Subsurface geological features, such as formations, aquifers, and reservoirs, can include porous rock or cavities, through which liquids and/or gases can be located. For example, reservoirs of water and hydrocarbons can be located in various subsurface geological features. Additionally, water and a number of gases can also be located in subsurface geological features. The location and structure of geological features in which subsurface liquids and/or gases are disposed can be challenging to model, especially as the depths below the surface increase, because of the variation that can be present in the characteristics of the rock that comprises subsurface geological features and because of the relatively large size of many subsurface geological features. Thus, entities that are interested in understanding the location and movement of gas and/or fluids through subsurface geological features typically rely on conventional methods of modeling subsurface geological formations that are often expensive and/or inaccurate.

### BRIEF DESCRIPTION OF THE DRAWINGS

Some implementations are illustrated by way of example and not limitation in the figures of the accompanying drawings.

FIG. 1 is an example framework to determine the movement of a substance within one or more subsurface geological features, according to one or more examples.

FIG. 2 is an example environment to obtain samples that can be analyzed to determine the flow of carbon-based gas within one or more subsurface geological features, according to one or more examples.

FIG. 3 is a diagram of an example framework to extract genetic material from samples obtained from subsurface geological features to determine the flow of carbon-based gas through the subsurface geological features, according to one or more examples.

FIG. 4 is a diagram showing that microbial compositions of fluid located in subsurface geological features change as the fluid is displaced by injected carbon dioxide, according to one or more examples.

FIG. 5 is a diagram illustrating the movement of carbon dioxide from a number of geological features over time determined based on monitoring changes in genetic material extracted from samples collected at a number of locations and depths, according to one or more examples.

FIG. 6 is a diagram illustrating the flow of carbon dioxide in a number of geological features based on analyzing genetic material of microbial communities disposed in fluids present in the number of geological features, according to one or more examples.

FIG. 7 is a diagram illustrating the detection of a fracture in caprock based on analyzing genetic material of microbial communities disposed in fluids present in the number of geological features, according to one or more examples.

FIG. 8 is a diagram illustrating the detection of carbon dioxide at a monitoring borehole or a producer borehole based on analyzing genetic material of microbial communities disposed in fluids present in the number of geological features, according to one or more examples.

FIG. 9 is a diagram illustrating the detection of the presence of carbon dioxide in an aquifer based on analyzing genetic material of microbial communities disposed in fluids present in the number of geological features, according to one or more examples.

FIG. 10 is a flow diagram of an example process to determine the flow of carbon-based gas through one or more subsurface geological features, according to one or more examples.

FIG. 11 is a chart showing the log of microbial communities located at a number of depths of one or more subsurface geological features.

FIG. 12 is a diagram of a subsurface model indicating the flow of carbon dioxide through one or more subsurface geological features.

FIG. 13 is a diagram showing a cross-section of a model of a number of geological features indicating microbial communities present in the number of subsurface geological features.

FIG. 14 is a diagram showing the impact on the movement of carbon dioxide through a number of subsurface geological features based on different carbon dioxide injection pressures.

FIG. 15 illustrates a diagrammatic representation of a machine in the form of a computer system within which a set of instructions may be executed for causing the machine to perform any one or more of the methodologies discussed herein, according to one or more examples.

### DETAILED DESCRIPTION

In the following description, for purposes of explanation, numerous specific details are set forth in order to provide an understanding of various implementations of the present subj ect matter. It will be evident, however, to those skilled in the art that various implementations may be practiced without these specific details.

Many entities have an interest in determining the characteristics of subsurface geological features. For example, hydrocarbon producers desire to understand the characteristics of subsurface geological features to identify the locations of hydrocarbons in the subsurface geological features and to extract the hydrocarbons. Additionally, governmental entities that are responsible for water quality and/or conservation have a need to determine possible sources of contamination of drinking water that may be located in or near various subsurface geological formations. In still other examples, entities that capture harmful gases, such as carbon dioxide (CO₂), and store the gases in subsurface geological features are also interested in understanding characteristics of subsurface geological features. In these situations, it can be desirable to understand the flow of carbon-based gas, such as CO₂, through a subsurface geological formation to determine the capacity for storing the carbon-based gases. Further, entities seeking to store CO₂ in subsurface geological features can be interested in determining whether the storage of CO₂ in a subsurface geological feature can cause ground water contamination.

In implementations described herein, the location of at least one of gases or liquids within subsurface geological formations can be determined. In one or more examples, genomic data extracted from at least one of solid samples or liquid samples obtained from a number of depths and at different locations along the surface can be analyzed. For example, one or more first wellbores can be drilled at one or more first locations. First samples that include at least one of solid material or liquid substances can be obtained from a number of first depths during and/or after the drilling of the one or more first wellbores. Additionally, one or more second wellbores can be drilled at one or more second locations that are a distance away from the one or more first wellbores. Second samples that include at least one of solid material or liquid substances can be obtained from a number of second depths during and/or after the drilling of the one or more second wellbores. In at least some examples, the second depths may correspond to the first depths. For example, the first depths may be approximately the same as the second depths, such that samples obtained from the one or more first boreholes are collected from depths that are the same as or similar to the depths from which the samples obtained from the one or more second boreholes are collected.

First genetic material can be extracted from the one or more first samples and second genetic material can be extracted from the one or more second samples. In one or more illustrative examples, the first genetic material and the second genetic material can comprise nucleic acids. The first genetic material can be analyzed to determine one or more first microbial communities present at the first depths. Additionally, the second genetic material can be analyzed to determine one or more second microbial communities present at the second depths. In various examples, the one or more second microbial communities can be analyzed with respect to the one or more first microbial communities to determine whether or not at least one of fluid or gas present at the one or more first locations is also present at the one or more second locations. In at least some examples, the one or more second microbial communities can be analyzed with respect to the one or more first microbial communities to determine an amount of movement of at least one of one or more gases or one or more fluids from the one or more first locations to the one or more second locations.

The first samples can be collected at one or more first times and one or more second samples can be collected at one or more second times. The one or more second times can be subsequent to the one or more first times. In one or more examples, the one or more first times can correspond to injection of a substance into the one or more first wellbores and the one or more second times can be subsequent to the injection of the substance into the one or more first wellbores. In one or more illustrative examples, the substance can include CO₂. In various examples, the CO₂ can be injected into the one or more first wellbores as part of a subsurface carbon storage project, such as a carbon capture and storage project or a carbon capture, utilization, and storage project.

Subsurface carbon storage can be used to mitigate carbon emissions. In at least some scenarios, subsurface carbon storage projects can be implemented with respect to hydrocarbon extraction operations. For example, subsurface carbon storage can be performed in enhanced oil recovery operations. Additionally, subsurface carbon storage can be performed in depleted hydrocarbon reservoirs. Further, subsurface carbon storage can take place in subsurface saline formations. In still additional situations, subsurface carbon storage can be performed in coal seams where the coal is unable to be extracted.

In many situations, subsurface carbon storage is employed to maximize carbon storage in subsurface geological features. The desire to maximize the amount of carbon stored is often balanced with the need to preserve the integrity of freshwater sources. Thus, integrity of caprock is a factor to consider in relation to subsurface carbon storage because as CO₂ is injected into subsurface storage locations, pressures within the subsurface storage locations can increase. In some cases, the pressure within the subsurface geological formations into which CO₂ is injected can cause fissures or fractures in the caprock. In these instances, CO₂ may leak into subsurface freshwater sources and cause contamination to take place. Additionally, a consideration in maximizing carbon storage in subsurface geological features is the amount of carbon storage space that is available in the subsurface geological features. However, it can be costly and challenging to gather the measurements needed to determine the size of a subsurface geological features as well as difficult to analyze the measurements in a manner that provides an accurate estimate of the amount of carbon storage space available in a given subsurface geological feature because the movement of CO₂ through geological formations can be difficult to model using existing techniques..

The analysis of genomic data extracted from samples collected from the one or more first boreholes and the one or more second boreholes can provide a high resolution data set that can be analyzed to accurately determine the location of CO₂ within a subsurface geological feature. By using the analysis of genetic material located at a number of subsurface depths extracted from samples obtained from the one or more first boreholes and the one or more second boreholes, the pressure buildup can be determined. In at least some cases, pressure mitigation techniques can be employed to decrease the pressure in a subsurface geological formation to preserve caprock integrity. Further, early detection of CO₂ contamination of freshwater aquifers can also be determined by analyzing genetic material extracted from samples collected from the one or more first boreholes and the one or more second boreholes. Early detection of the presence of stored CO₂ in freshwater aquifers can help to minimize the contamination and preserve the freshwater located in the aquifers. In additional scenarios, genetic material extracted from samples collected from the one or more first boreholes and the one or more second boreholes can be analyzed to determine the location and/or movement of CO₂ within subsurface geological features in order to determine an amount of capacity for carbon storage in the subsurface geological features and the amount of remaining capacity available to store additional amounts of CO₂.

Genetic material extracted from samples collected from the one or more first boreholes and the one or more second boreholes can provide a high resolution and relatively inexpensive data source for locating and tracking one or more carbon-based gases within subsurface geological features because the genetic material can indicate the presence of various microbial communities found within the subsurface geological features. In various examples, microbial communities can be found in the pores and fracture spaces that are located at a number of depths from relatively shallow freshwater aquifers to reservoirs that are many kilometers below the surface. These microbial communities reside in relatively extreme, but stable environments. Additionally, because these microbial communities are found in environments having a variety of pressure and temperature conditions as well as in numerous fluid and rock types, the microbial communities have adapted to particular environments. As a result, the microbial communities located in different depths and in different subsurface geological features have compositions with sufficient diversity to be differentiated from one another. For example, microbial diversity can be sufficient to distinguish between different vertically arranged subsurface geological features. Further, the microbial communities located with respect to the one or more first boreholes can have an amount of diversity with respect to the microbial communities related to the one or more second boreholes to differentiate the samples obtained from the one or more first boreholes and the samples obtained from the one or more second boreholes.

Due to the amount of diversity among microbial communities located at different subsurface depths and in different subsurface geological features, changes in microbial communities can indicate movement of at least one of liquid or gas through the subsurface geological features. For example, as CO₂ is injected into a geological feature, the CO₂ can displace fluid located in the subsurface geological feature. The movement of the displaced fluid from a first location of the subsurface geological feature to a second location of the subsurface geological feature can cause changes in microbial communities downstream from the injection site. Thus, as changes to the microbial communities in different portions of a subsurface geological feature are detected, the movement of the CO₂ within the subsurface geological feature can also be determined. In various examples, the contribution of different sources of microorganisms at a portion of a subsurface geological feature where the one or more second wellbores are located can be determined to generate a fluid contribution log at a given location of the subsurface geological feature. The fluid contribution log can indicate movement of CO₂ within the subsurface geological features as the contribution of the microbial community at a location downstream from the CO₂ injection site changes.

As the changes to the microbial communities at a number of locations of a subsurface geological feature are detected, CO₂ movement models can be generated. The CO₂ movement models can then be used to determine whether pressure mitigation measures are needed or whether a caprock breach has occurred. The CO₂ movement models can also be used to determine an amount of carbon storage capacity remaining in a subsurface geological feature or whether contamination of an aquifer has taken place. Thus, the collection and analysis of genetic material obtained from a number of locations of a geological feature can provide accurate insight into subsurface conditions that can be used to effectively and safely store carbon in subsurface geological features.

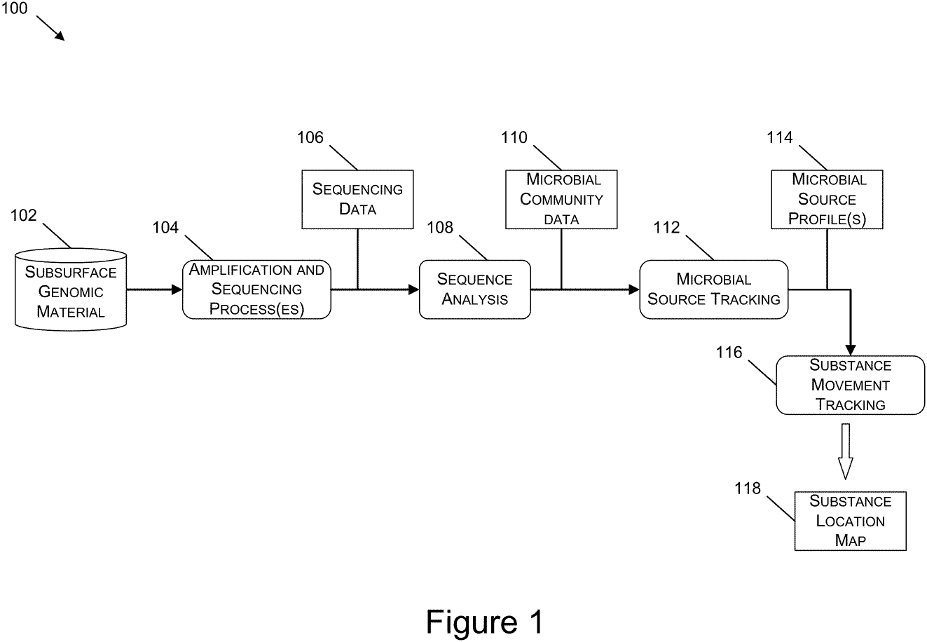

FIG. 1 is an example framework 100 to determine the movement of a substance within one or more subsurface geological features, according to one or more examples. The framework 100 can include subsurface genetic material 102. The subsurface genetic material 102 can include at least one of deoxyribonucleic acid (DNA) or ribonucleic acid (RNA) extracted from samples collected from one or more wellbores. The subsurface genetic material 102 can correspond to a number of different biological organisms. In one or more illustrative examples, the subsurface genetic material 102 can corresponds to a number of microorganisms. In these scenarios, the subsurface genetic material 102 can correspond to genetic material of microbial colonies comprised of thousands of microorganisms, tens of thousands of microorganisms, hundreds of thousands of microorganisms, millions of microorganisms, or more. The microorganisms included in the microbial colonies can be classified according to a number of different phyla, a number of different families, a number of different genera, a number of different species, or one or more combinations thereof.

The subsurface genetic material 102 can undergo one or more amplification and sequencing processes at 104. The one or more amplification processes can increase a quantity of nucleic acids of a number of microorganisms. In various examples, the one or more amplification and sequencing processes can include performing one or more polymerase chain reaction (PCR) processes. In one or more additional examples, the one or more amplification and sequencing processes can include an isothermal amplification process, such as a multiple displacement amplification (MDA) process. Amplification of the subsurface genetic material 102 can generate an amplification product that includes hundreds of thousands of copies of individual nucleic acids included in the subsurface genetic material 102, millions of copies of individual nucleic acids included in the subsurface genetic material 102, tens of millions of copies of individual nucleic acids included in the subsurface genetic material 102, or more. The amplification product can include a total of hundreds of millions of nucleic acid molecules, a total of billions of nucleic acid molecules, or more.

The one or more amplification and sequencing processes can also include determining sequences of nucleotides that comprise the nucleic acid molecules included in the amplification product. For example, the one or more amplification and sequencing processes 104 can generate sequencing data 106. The sequencing data 106 can include sequence reads that include strings of letters that indicate nucleotides at a given position of nucleic acids derived from the subsurface genetic material 102. Individual sequencing reads can represent a determination of which of the four nucleotides—A, G, C, and T (or U)-in a strand of DNA (or RNA) is present at a given position of a nucleic acid.

The sequencing data 106 can undergo sequence analysis at operation 108. The sequence analysis can determine sequence reads that correspond to nucleic acids of a number of microorganisms. For example, the sequence analysis performed at operation 108 can determine respective portions of the sequence reads included in the sequencing data 106 that correspond to DNA of individual microorganisms located in an environment from which the subsurface genetic material 102 was collected. To illustrate, the sequence analysis performed at operation 108 can determine first sequence reads that correspond to a first microorganism, second sequence reads that correspond to a second microorganism, third sequence reads that correspond to a third microorganism, and so forth until sequence reads of thousands of microorganisms up to millions of microorganisms are identified. In one or more illustrative examples, the sequence analysis performed at operation 108 can analyze sequence reads with regard to reference nucleic acid sequences where individual reference nucleic acid sequences correspond to individual microorganisms. In at least some examples, the sequence analysis can include determining an amount of identity or an amount of homology between sequence reads included in the sequencing data 106 and the reference sequences that correspond to individual microorganisms. In scenarios where a sequence read has at least a threshold amount of identity with respect to a reference sequence, the sequence read can be identified as corresponding to the microorganism associated with the reference sequence.

The sequence analysis performed at operation 108 can generate microbial community data 110. The microbial community data 110 can correspond to microorganism that are identified based on the subsurface genetic material 102. In various examples, the microbial community data 110 can include identifiers for the microorganisms that correspond to the subsurface genetic material 102. In one or more illustrative examples, microorganisms can be identified based on an operational taxonomic unit classification system.

At operation 112, microbial source tracking can be performed using the microbial community data 110 to produce one or more microbial source profiles 114. In one or more examples, the microbial source tracking can identify a source of microorganisms that are located at a given location that is different from a reference location that corresponds to the source. In various examples, a first portion of the subsurface genetic material 102 can correspond to first samples obtained from a first wellbore at a first location. Additionally, a second portion of the subsurface genetic material 102 can correspond to second samples obtained from a second wellbore at a second location. In one or more illustrative examples, the first wellbore can be used to inject a substance into one or more subsurface geological features. To illustrate, the first wellbore can be used to inject CO₂ into one or more subsurface geological features. In this way, the first wellbore is a reference location because the first wellbore corresponds to an initial point of entry of the substance into the one or more subsurface geological features.

The second wellbore can be located at a location that is different from the injection point of the substance. As a result, the second wellbore can serve as a monitoring point to determine an amount of movement of the substance from the initial injection point at the first wellbore. Thus, the second samples can be collected from the second wellbore at one or more times subsequent to the injection of the substance at the first wellbore in order to determine how much the substance has moved from the location of the first wellbore with respect to the location of the second wellbore.

The microbial source tracking at operation 112 can analyze a first portion of the microbial community data 110 that corresponds to the first portion of the subsurface genetic material 102 collected from the first wellbore with respect to a second portion of the microbial community data 110 that corresponds to the second portion of the subsurface genetic material 102 collected from the second wellbore. In various examples, the microbial source tracking can determine the sources of microorganisms present in the samples obtained from the second wellbore based on the microbial communities present in the samples collected from the second wellbore. In one or more examples, the microbial source tracking may indicate that a source of a portion of the microbial community data 110 is a subsurface geological feature in which the second wellbore has been drilled. In one or more additional examples, the microbial source tracking may also indicate that an additional source of an additional portion of the microbial community data 110 is an additional subsurface geological feature in which the first wellbore has been drilled. In this way, the one or more microbial source profiles 114 can indicate a first amount of subsurface genetic material 102 collected from the second samples obtained from the second wellbore that have a first source corresponding to a first subsurface geological feature in which the first wellbore is drilled and can also indicate a second amount of the subsurface genetic material 102 collected from the second samples obtained from the second wellbore that have a second source corresponding to a second subsurface geological features in which the second wellbore is drilled.

The framework 100 can also include performing substance movement tracking at operation 116 using the one or more microbial source profiles 114. In one or more examples, the substance movement tracking performed at operation 116 can generate a substance location map 118. In various examples, the substance location map 118 can indicate where the substance has traveled since being injected into the first wellbore. The substance movement tracking can generate the substance location map 118 by analyzing the contribution of individual microbial communities from the respective sources indicated by the one or more microbial source profiles. For example, the greater the amount of subsurface genetic material 102 present in a given location that is attributed to a respective source, the farther a substance has traveled away from the source. To illustrate, and continuing with the example from above, an amount of the microorganisms found in the first subsurface geological feature in which the first wellbore is drilled relative to an amount of the microorganisms found in the second geological feature in which the second wellbore is drilled can indicate an amount that CO₂ has moved away from the first geological feature toward the second geological feature. In at least some examples, the distance that the CO₂ has moved away from the first geological feature can be shown on the substance location map 118.

The substance location map 118 can be used to determine whether or not the substance has moved to an undesirable subsurface location. The substance location map 118 can also be used to determine one or more measures to perform to stop or modify the movement of the substance within one or more subsurface geological formations. Further, the substance location map 118 can indicate characteristics of subsurface geological features, such as fissures, fractures, or other indicators of weaknesses in the integrity of subsurface geological features. For example, in situations where the substance location map 118 indicates movement of CO₂ away from an injection site, the substance location map 118 can indicate whether or not the injected CO₂ has moved into a freshwater aquifer and started to contaminate a portion of the freshwater included in the aquifer. In one or more additional examples, the substance location map 118 can indicate fractures in caprock by showing that the CO₂ has breached the caprock and moved into a subsurface geological feature above the caprock. In one or more further examples, the substance location map 118 can indicate pressure increases in one or more subsurface geological formations caused by the injection of CO₂ that can result in changes to one or more subsurface geological formations that can impact one or more resources of the one or more subsurface geological formations.

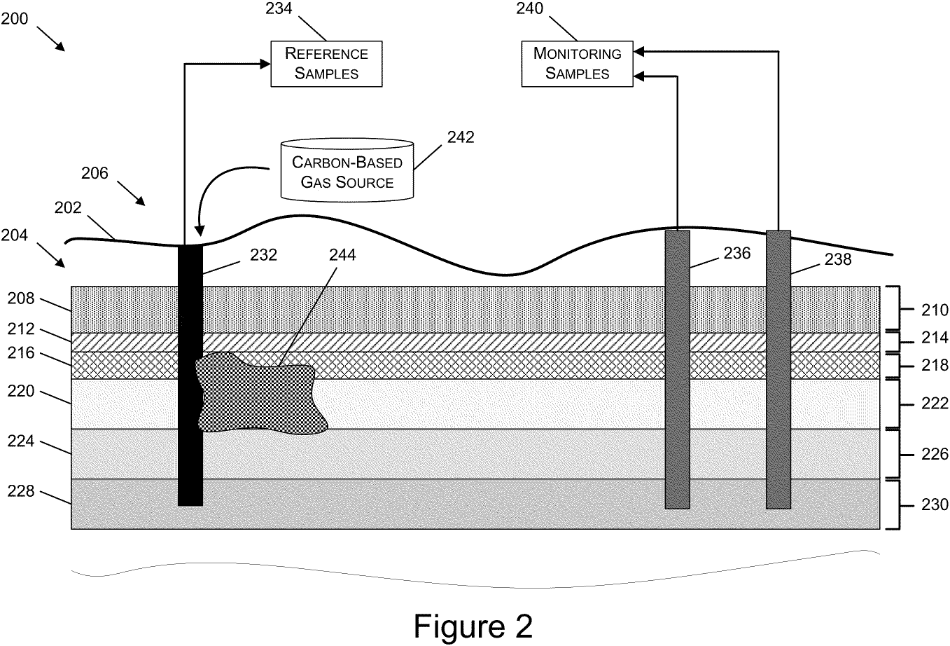

FIG. 2 is an example environment 200 to obtain samples that can be analyzed to determine the flow of carbon dioxide within one or more subsurface geological features, according to one or more examples. The environment 200 can include a surface 202, a subsurface region 204, and an above surface region 206. The surface 202 can be located at an interface between land and air. In these scenarios, the above surface region 206 can be comprised of air. In one or more additional examples, the surface 202 can be located at an interface between land and water. In these instances, the above surface region 206 can include a body of water, such as an ocean, a sea, a lake, or a river.

The subsurface region 204 can include a number of geological features. For example, the subsurface region 204 can include a first geological feature 208 found at first depths 210 and a second geological feature 212 found at second depths 214. Additionally, the subsurface region 204 can include a third geological feature 216 at third depths 218 and a fourth geological feature 220 at fourth depths 222. Further, the subsurface region 204 can include a fifth geological feature 224 at fifth depths 226 and a sixth geological feature 228 at sixth depths 230. In various examples, the geological features 208, 212, 216, 220, 224, 228 located in the subsurface region 204 can be found from a few meters below the surface 202 up to several kilometers below the surface 202. In various examples, the depths 210, 214, 218, 222, 226, 230 can individually range from several meters up to one or more kilometers.

The geological features 208, 212, 216, 220, 224, 228 can have distinctive features such that the individual geological features 208, 212, 216, 220, 224, 228 are distinguishable from one another. For example, the composition of rock and/or soil of the individual geological features 208, 212, 216, 220, 224, 228 can be used to distinguish the geological features 208, 212, 216, 220, 224, 228 from one another. To illustrate, at least one of the porosity, hardness, mineral composition, or molecular composition of the rocks and/or soil of the individual geological features 208, 212, 216, 220, 224, 228 can be used to distinguish the geological features 208, 212, 216, 220, 224, 228 from one another. In addition, at least one of the composition or amount of fluid and/or gas present in the individual geological features 208, 212, 216, 220, 224, 228 can distinguish the geological features 208, 212, 216, 220, 224, 228 from one another. In one or more illustrative examples, at least a portion of the geological features 208, 212, 216, 220, 224, 228 can include very little fluid, while other geological features 208, 212, 216, 220, 224, 228 can be composed of relatively large amounts of fluid or be composed primarily of fluid. In scenarios, where at least a portion of the geological features 208, 212, 216, 220, 224, 228 comprise a relatively large amount of fluid, those geological features can be distinguishable based on the composition of the fluids located in them. In one or more additional illustrative examples, one or more of the geological features 208, 212, 216, 220, 224, 228 can comprise freshwater and one or more additional geological features 208, 212, 216, 220, 224, 228 can comprise water having a relatively high concentration of salt, such as brine.

In still further illustrative examples, the geological features 208, 212, 216, 220, 224, 228 can be differentiated based on the microorganisms present in the individual geological features 208, 212, 216, 220, 224, 228. In various examples, as the geological features 208, 212, 216, 220, 224, 228 formed over thousands of years, the microorganisms present in the geological features 208, 212, 216, 220, 224, 228 have adapted to the characteristics and environment of the individual geological features 208, 212, 216, 220, 224, 228. Thus, the distinguishing physical, chemical, and environmental characteristics of the geological features 208, 212, 216, 220, 224, 228 can result in communities of microorganisms present in the individual geological features 208, 212, 216, 220, 224, 228 that are also distinguishable from one another.

In one or more illustrative examples, the first geological feature 208 can include a first rock formation and the second geological feature 212 can include a second rock formation. The first rock formation and the second rock formation can be comprised of one or more rock materials having one or more degrees of porosity. In one or more additional illustrative examples, the first geological feature 208 can include a rock formation and the second geological feature 212 can include a freshwater aquifer. In one or more additional illustrative examples, the third geological feature 216 can include caprock. The caprock can be less porous than rock formations found above the caprock. In at least some examples, the third geological feature 216 can be comprised of caprock that is impermeable in at least some locations with respect to a number of gases and/or liquids. In one or more further illustrative examples, the fourth geological features 220 can include a first reservoir, the fifth geological feature 224 can include a second reservoir, and the sixth geological feature 228 can include a third reservoir. In still other illustrative examples, at least one of the fourth geological feature 220, the fifth geological feature 224, or the sixth geological feature 228 can comprise a reservoir and at least another one of the fourth geological feature 220, the fifth geological feature 224, or the sixth geological feature 228 can comprise a rock formation. In various examples when at least one of the fourth geological feature 220, the fifth geological feature 224, or the sixth geological feature 228 includes a reservoir, the reservoir can include one or more fluids. The one or more fluids can include brine. The one or more fluids can also include one or more hydrocarbons.

In various examples, individual geological features 208, 212, 216, 220, 224, 228 can have respective environmental conditions that can be characterized according to at least one of temperature, pressure, pH, or one or more additional physical characteristics. Additionally, individual geological features 208, 212, 216, 220, 224, 228 can support microbial communities that are different from one another. For example, the first geological feature 208 can be a first habitat for a first microbial community and the second geological feature 212 can be a second habitat for a second microbial community. In addition, the third geological feature 216 can be a habitat for a third microbial community and the fourth geological feature 220 can be a habitat for a fourth microbial community. The fifth geological feature 224 can be a habitat for a fifth microbial community and the sixth geological feature 228 can be a habitat for a sixth microbial community.

The environment 200 can also include a first wellbore 232. The first wellbore 232 can be produced using heavy drilling equipment that can drill up to several kilometers beneath the surface 202. The first wellbore 232 can pass from the surface 202 through a number of subsurface geological features, such as the first geological feature 208, the second geological feature 212, the third geological features 216, the fourth geological feature 220, the fifth geological feature 224, and the sixth geological feature 228.

A number of reference samples 234 can be collected from the first wellbore 232. The reference samples 234 can include at least one of liquid, gas, or solid material obtained from one or more locations within the first wellbore 232. In one or more examples, the reference samples 234 can include material extracted from the first geological feature 208, the second geological features 212, the third geological feature 216, the fourth geological feature 220, the fifth geological feature 224, the sixth geological feature 228, or one or more combinations thereof.

The environment 200 can also include a second wellbore 236 and a third wellbore 238. The second wellbore 236 and the third wellbore 238 can span depths that are similar to those of the first wellbore 232. In one or more examples, at least one of the second wellbore 236 or the third wellbore 238 can be present in at least a portion of the geological features 208, 212, 216, 220, 224, 228. Monitoring samples 240 can be collected from at least one of the second wellbore 236 or the third wellbore 238. The monitoring samples 240 can include at least one of liquid, gas, or solid material obtained from one or more locations within at least one of the second wellbore 236 or the third wellbore 238. In one or more examples, the monitoring samples 240 can include material extracted from the first geological feature 208, the second geological features 212, the third geological feature 216, the fourth geological feature 220, the fifth geological feature 224, the sixth geological feature 228, or one or more combinations thereof.

The environment 200 can also include a carbon-based gas source 242. The carbon-based gas source can provide one or more carbon-based gases that are injected into the first wellbore 232. In one or more illustrative examples, the one or more carbon-based gases can include at least one of CO₂, methane (CH₄), or carbon monoxide (CO). The one or more carbon-based gases from the carbon-based gas source 242 can be injected into the first wellbore 232 at pressures that are above atmospheric pressure. For example, one or more carbon-based gases can be injected into the first wellbore 232 at pressures of several megapascals (MPa), such as from one MPa to 20 MPa, from 2 MPa to 15 MPa, from 4 MPa to 12 MPa, from 2 MPa to 10 MPa, or from 8 MPa to 12 MPa. As the one or more carbon-based gases are injected into the first wellbore 232, the one or more carbon-based gases can spread into the geological features 208, 212, 216, 220, 224, 228. In at least some examples when the one or more carbon-based gases include CO₂, at least a portion of the CO₂ injected into the first wellbore 232 can become supercritical CO₂. That is, the CO₂ injected into the first wellbore 232 can be present in at least a portion of the geological features 208, 212, 216, 220, 224, 228 at temperatures at or above the critical temperature of CO₂ (31.0° C.) and at or above the critical pressure of CO₂ (7.3773 MPa).

In one or more illustrative examples, one or more carbon-based gases can be injected into the first wellbore 232 as part of enhanced oil recovery operations. For example, the one or more carbon-based gases can be injected into the first wellbore 232 from the carbon-based gas source 242 to cause an increased amount of hydrocarbons to be extracted from one or more of the geological features 208, 212, 216, 220, 224, 228. In various examples, the carbon-based gas source 242 can include one or more carbon-based gases produced during the extraction of hydrocarbons from the one or more geological features 208, 212, 216, 220, 224, 228. In one or more additional examples, at least a portion of the geological features 208, 212, 216, 220, 224, 228 were used in previous hydrocarbon recovery operations and the one or more carbon-based gases injected into the first wellbore 232 can be stored in portions of the geological features 208, 212, 216, 220, 224, 228 from which hydrocarbons were extracted. In one or more further examples, the one or more carbon-based gases can be injected into at least a portion of the geological features 208, 212, 216, 220, 224, 228 that store at least one of freshwater or saltwater.

In one or more examples, genetic material extracted from the monitoring samples 240 can be used to determine a composition of microorganisms present in one or more of the geological features 208, 212, 216, 220, 224, 228 at the positions of at least one of the second wellbore 236 or the third wellbore 238. Additionally, genetic material extracted from the reference samples 234 can also be analyzed to determine a composition of microorganisms present in one or more of the geological features 208, 212, 216, 220, 224, 228 at the position of the first wellbore 232. The compositions of the microorganisms in one or more of the geological features 208, 212, 216, 220, 224, 228 at the position of the second wellbore 236 and/or the position of the third wellbore 238 can be analyzed with respect to the composition of microorganisms in one or more of the geological features 208, 212, 216, 220, 224, 228 at the position of the first wellbore 232 to determine a region 244 that corresponds to the position of the one or more carbon-based gases within one or more of the geological features 208, 212, 216, 220, 224, 228.

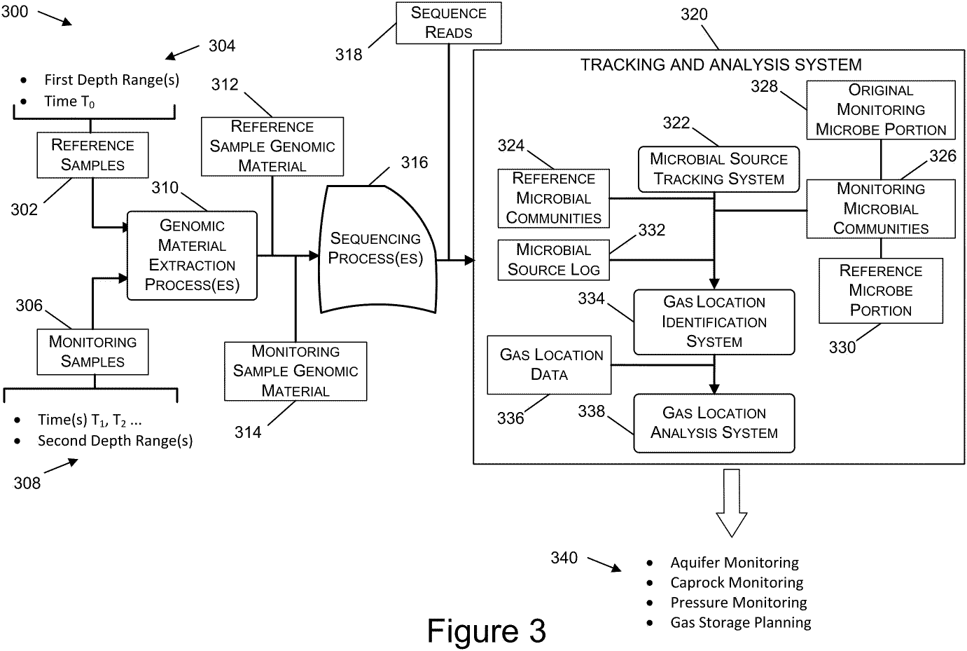

FIG. 3 is a block diagram of an example framework 300 to extract genetic material from samples obtained from subsurface geological features to determine the flow of carbon dioxide through the subsurface geological features, according to one or more examples. The framework 300 can include reference samples 302. The reference samples 302 can include at least one of solid material, liquid material, or gaseous material collected from one or more reference wellbores. In one or more examples, the one or more reference wellbores can include at least one CO₂ injection site. The reference samples 302 can be extracted from different subsurface depths. In various examples, the reference samples 302 can be extracted from different geological features through which a reference wellbore passes. In one or more illustrative examples, the reference samples 302 can correspond to first conditions 304. The first conditions 304 can indicate that the reference samples 302 were collected at one or more times, such as an initial time, T₀. The first conditions 304 can also indicate first depth ranges from which the reference samples were collected. In at least some cases, the first depth ranges can be used to determine one or more subsurface geological features from which the reference samples 302 were collected. In at least some examples, the reference samples 302 can correspond to at least a portion of the reference samples 234 described with respect to FIG. 2.

The framework 300 can also include monitoring samples 306. The monitoring samples 306 can include at least one of solid material, liquid material, or gaseous material collected from one or more monitoring wellbores. In one or more examples, the one or more monitoring wellbores can be located a respective distance from a CO₂ injection site, such as at least 0.5 kilometers (km), at least 1 km, at least 2 km, at least 3 km, at least 5 km, at least 8 km, or at least 10 km. In various examples, the one or more monitoring wellbores can include abandoned hydrocarbon extraction wellbores.

The monitoring samples 306 can be extracted from different subsurface depths. In various examples, the monitoring samples 306 can be extracted from different geological features through which a monitoring wellbore passes. In one or more examples, the monitoring samples 306 can correspond to second conditions 308. The second conditions 308 can indicate that the monitoring samples 306 were collected at one or more times, such T1, T2 .... T1 and T2 can be subsequent to T0 of the first conditions 304. In one or more illustrative examples, T0 can be a time before injection of CO₂ into the reference wellbore and T1, T2 ... can be times that are subsequent to injection of CO₂ into the reference wellbore. In various examples, the monitoring samples 306 can be collected periodically. The second conditions 308 can also indicate second depth ranges from which the monitoring samples were collected. In at least some cases, the second depth ranges can be used to determine one or more subsurface geological features from which the monitoring samples 306 were collected. In at least some examples, the monitoring samples 306 can correspond to at least a portion of the monitoring samples 240 described with respect to FIG. 2.

In addition, the framework 300 can include one or more genetic material extraction processes 310. The one or more genetic material extraction processes 310 can be used to extract reference sample genetic material 312 from the reference samples 302. The one or more genetic material extraction processes 310 can also be used to extract monitoring sample genetic material 314 from the monitoring samples 306. In one or more examples, the one or more genetic material extraction processes 310 can separate nucleic acids from other, unwanted cellular and sample matter in a way to make the genetic material suitable for additional processing and analysis. For example, the one or more genetic material extraction processes 310 can include mechanical processes, such as at least one of bead beating, sonicating, or performing one or more freezing and thawing cycles. The one or more genetic material extraction processes 310 can include chemical processes that use at least one of detergents, acids, bases, enzymes, or other organic or inorganic chemicals. In various examples, the one or more genetic material extraction processes 310 can include binding and elution from silica matrices, washing and precipitation by organic or inorganic chemicals, electroelution or electrophoresis, or other methods capable of isolating genetic material from other material included in the reference samples 302 and other material included in the monitoring samples 306. The reference sample genetic material 312 can include at least one of DNA molecules or RNA molecules extracted from the reference samples. Additionally, the monitoring sample genetic material 314 can include at least one of DNA304 molecules or RNA molecules extracted from the monitoring samples 306.

The reference sample genetic material 312 and the monitoring sample genetic material 314 can undergo one or more sequencing processes 316. The one or more sequencing processes 316 can amplify the nucleic acid molecules included in the reference sample genetic material 312 and in the monitoring sample genetic material 314. For example, the one or more sequencing processes 316 can generate thousands, tens of thousands, up to millions of copies or more of individual nucleic acids included in the reference sample genetic material 312 and nucleic acids included in the monitoring sample genetic material 314. Amplification of nucleic acid molecules included in the reference sample genetic material 312 and the monitoring sample genetic material 314 can be performed using amplification reagents that can include primers used in the amplification process, buffer solutions, one or more enzymes, nucleotides, combinations thereof, and so forth. The one or more sequencing processes 316 can also determine nucleotide sequences of molecules that undergo one or more amplification and sequencing processes. The nucleotide sequences can indicate a nucleotide present at a given position of a nucleic acid molecule. The one or more sequencing processes 316 can be performed by one or more machines. The one or more machines can perform one or more polymerase chain reaction (PCR) processes. In one or more additional examples, one or more machines performing the one or more sequencing processes 316 can perform one or more multiple displacement amplification (MDA) processes. In at least some examples, the one or more sequencing processes 316 can include one or more high-throughput sequencing processes.

With regard to scenarios where one or more PCR processes are performed in relation to the one or more sequencing processes 316, individual PCR reactions included in the one or more sequencing processes 316 can have at least three main components: the template, the primers, and enzymes. The template is a single- or double-stranded molecule containing the (sub)sequence of nucleotides to be amplified. The primers are short strands (e.g., less than 40 nucleotides) that define the beginning and end of the region to be amplified. The enzymes include polymerases and thermostable polymerases such as DNA polymerase, RNA polymerase and reverse transcriptase. The enzymes create double-stranded polynucleotides from a single-stranded template by filling in″ complementary nucleotides one by one through addition of nucleoside triphosphates, starting from a primer bound to that template. PCR happens in cycles, each of which doubles the number of templates in a solution. The process can be repeated until the desired number of copies is created.

In at least some examples, the one or more sequencing processes 316 can be performed with respect to one or more genomic regions. In one or more illustrative examples, the one or more sequencing processes can be performed such that one or more genomic regions present in the reference sample genetic material 312 and in the monitoring sample genetic material 314 are amplified. For example, the one or more sequencing processes 316 can be performed to amplify genomic regions of the reference sample genetic material 312 and the monitoring sample genetic material 316 that correspond to at least a portion of the 16S rRNA gene. In one or more illustrative examples, one or more regions of the 16s rRNA gene can be targeted as part of the one or more sequencing processes 316, such as the V3, V4, and/or V5 subregions. In one or more additional illustrative examples, one or more regions of the 18s rRNA gene can be targeted as part of the one or more sequencing processes 316.

The one or more sequencing processes 316 can generate sequence reads 318. The sequence reads 318 can represent single stranded or double stranded nucleic acid molecules generated by the one or more sequencing processes 316. The sequence reads 318 can have lengths from about 50 nucleotides to about 500 nucleotides, from about 75 nucleotides to about 300 nucleotides, from about 100 nucleotides to about 200 nucleotides, from about 50 nucleotides to about 150 nucleotides, or from about 50 nucleotides to about 250 nucleotides. The sequence reads 318 can be stored in a data file. For example, the sequence reads 318 can be stored in one or more FASTQ files, one or more binary alignment map (BAM) files, or one or more variant call format (VCF) files.

The environment 300 can also include a tracking and analysis system 320. The tracking and analysis system 320 can analyze the sequence reads 318 to determine subsurface geological features that are sources of the genetic material represented by the sequence reads 318. The tracking and analysis system 320 can also analyze the sequence reads 318 to determine movement of one or more carbon-based gases through the subsurface geological features. The movement of carbon-based gases through the subsurface geological features can be used to determine strategies and procedures around the storage of carbon-based gases in subsurface geological features.

The tracking and analysis system 320 can include a microbial source tracking system 322. The microbial source tracking system 322 can analyze the sequence reads 318 to determine subsurface geological features that correspond to sources of nucleic acids represented by the sequence reads 318. In one or more examples, the microbial source tracking system 322 can analyze a portion of the sequence reads 318 that corresponds to the reference sample genetic material 312 to determine reference microbial communities 324. The reference microbial communities 324 can include microorganisms present in subsurface geological features at a location of a carbon-based gas injection site. The microbial source tracking system 322 can also analyze a portion off the sequence reads 318 that corresponds to the monitoring sample genetic material 314 to determine monitoring microbial communities 326. The monitoring microbial communities 328 can include microorganisms present in subsurface geological features at a location of one or more monitoring wellbores.

The microbial source tracking system 322 can determine the reference microbial communities 324 and the monitoring microbial communities 326 by analyzing the sequence reads 318 with respect to genomic sequences of microorganisms. In at least some examples, a genomic sequence of the microorganisms analyzed by the microbial source tracking system 322 can correspond to at least a portion of the genome of the microorganism. For example, the microbial source tracking system 322 can analyze the sequencing reads 318 with respect to the genomic sequences of the microorganisms to determine an amount of identity between the sequence reads 318 and the genomic sequences of the microorganisms. In scenarios where an amount of identity between a sequence read 318 and at least a portion of a genome of a microorganism is at least threshold amount of identity, the microbial source tracking system 322 can determine that the sequence read 318 corresponds to the microorganism.

The microbial source tracking system 322 can also determine a location that corresponds to the sequence reads 318. In one or more examples, the location that corresponds to an individual sequence read 318 can be related to a sample from which the individual sequence read 318 was derived. For example, samples can be labeled according to the wellbore from which the samples were collected. Additionally, samples can be labeled according to depths from which the samples were collected. In various examples, the reference samples 302 can be labeled as being collected from an injection site of carbon-based gases in addition to the depths from which the reference samples 302 were collected. The monitoring samples 306 can be labeled as being collected from one or more monitoring sites and the depths from which the monitoring samples 306 were collected.

In at least some examples the one or more sequencing processes 316 can include adding bar codes or other identifiers to the nucleic acids included in the reference sample genetic material 312 and the nucleic acids included in the monitoring sample genetic material 314. The bar codes or other identifiers can indicate at least one of a location or depth corresponding to the respective sample from which the individual nucleic acids were derived. In various examples, the bar codes or other identifiers can include nucleotide sequences that individually identify a location and a depth from which the reference samples 302 and the monitoring samples 306 were collected. In one or more illustrative examples, the bar codes or other identifiers can comprise from 2 nucleotides to 20 nucleotides, from 4 nucleotides to 15 nucleotides, from 6 nucleotides to 10 nucleotides, from 5 nucleotides to 15 nucleotides, or from 4 nucleotides to 12 nucleotides.

In one or more examples, the microbial source tracking system 322 can determine a portion of the sequence reads 318 that correspond to reference microbial communities 324 by analyzing the sequence reads 318 with respect to bar codes or other identifiers that correspond to the reference samples 302. In one or more additional examples, the microbial source tracking system 322 can determine a portion of the sequence reads 318 that correspond to the monitoring microbial communities 326 by analyzing the sequence reads 318 with respect to bar codes or other identifiers that correspond to the monitoring samples 306.

In various examples, the microbial source tracking system 322 can determine an abundance of microorganisms that correspond to a given location and one or more depths. For example, the microbial source tracking system 322 can determine a number of sequence reads 318 that correspond to individual microorganisms. In at least some examples, the microbial source tracking system 322 can determine an abundance of an individual microorganism based on the number of sequence reads 318 that correspond to the individual microorganism. In one or more additional examples, the microbial source tracking system 322 can determine a relative abundance of microorganisms at a location and depth by analyzing the number of sequence reads 318 that correspond to an individual microorganism in relation to a number of sequence reads 318 of one or more additional microorganisms. In one or more illustrative examples, the one or more additional microorganisms can include one or more additional microorganisms that correspond to at least one of a location or depth of the individual microorganism.

The microbial source tracking system 322 can also analyze the sequence reads 318 to determine one or more classifications of microorganisms that correspond to the sequence reads 318. The one or more classifications can correspond to a taxonomic system that organizes microorganisms. In one or more examples, the taxonomic system can organize microorganisms according to at least one of phylum, family, genus, or species. In one or more illustrative examples, the microbial source tracking system 322 can determine an operational taxonomic unit (OTU) that corresponds to individual microorganisms that corresponds to the sequence reads 318. In one or more examples, the microbial source tracking system 322 can determine an OTU that includes a number of microorganisms that are organized according to an amount of similarity between genomic sequences of the microorganisms. In various examples, the reference microbial communities 324 and the monitoring microbial communities 326 can indicate, for individual microorganisms, a location of the individual microorganism, a depth of the individual microorganism, an OTU of the individual microorganism, and an abundance of the individual microorganism.

In one or more examples, the monitoring microbial communities 328 can include microorganisms from the one or more monitoring wellbores and also microorganisms from an injection site wellbore. In one or more illustrative examples, the monitoring microbial communities 326 can include an original monitoring microbe portion 328 and a reference microbe portion 330. In various examples, when one or more carbon-based gases are injected into one or more subsurface geological features using the injection site wellbore, fluid in the subsurface geological features at the injection site can migrate toward the one or more monitoring wellbores. In these scenarios, the composition of the original microbial communities in the one or more subsurface geological features at the one or more monitoring wellbores can be different from the composition of the microbial communities at the locations of the one or more monitoring wellbores after the injection of carbon-based gases. To illustrate, as the fluid from the subsurface geological features migrates from the location of the injection site toward the location of one or more monitoring wellbores, the microbial communities present in the subsurface geological features at the location of the one or more monitoring wellbores is a mixture of the original microbial communities present in the subsurface geological features at the location of the one or more monitoring wellbores and at least a portion of the microbial communities present in the subsurface geological features at the location of the carbon-based gas injection site. In the illustrative example of FIG. 3, the original monitoring microbe portion 328 can correspond to the microbial communities present in one or more subsurface geological features at the location of one or more monitoring wellbores prior to injection of the carbon-based gas into the injection wellbore and the reference microbe portion 330 can indicate the contribution to the monitoring microbial communities of the microorganisms present in fluid that has moved toward the location of the one or more monitoring wellbores from the location of the injection wellbore in response to the injection of carbon-based gas into the injection wellbore.

The microbial source tracking system 322 can separate the original monitoring microbe portion 328 from the reference microbe portion 330 in the monitoring microbial communities 326. For example, the microbial source tracking system 322 can analyze the microorganisms present in the monitoring microbial communities 326 in relation to the microorganisms present in the reference microbial communities 324. In various examples, the microbial source tracking system 322 can filter the microorganisms present in the reference microbial communities 324 from the monitoring microbial communities 326 to determine the reference microbe portion 330. In these scenarios, a remainder of the monitoring microbial communities 326 can correspond to the original monitoring microbe portion 328.

In at least some examples, the microbial source tracking system 322 can also determine one or more geological features at the location of the injection wellbore that correspond to the reference microbe portion 330. In this way, the microbial source tracking system 322 can determine a source of microorganisms present in the reference microbe portion 330. To illustrate, the microbial source tracking system 322 can analyze the microorganisms present in the reference microbe portion 330 with respect to the microorganisms present in the reference microbial communities 324 with respect to a number of subsurface geological features at the location of the injection wellbore. The microbial source tracking system 322 can then, for individual subsurface geological features at the location of the injection wellbore, determine the microorganisms included in the reference microbe portion 330 that correspond to the individual subsurface geological features. In various examples, the microbial source tracking system 322 can also determine an abundance of microorganisms of the reference microbe portion 330 by determining a number of the sequence reads 318 that correspond to the genomic sequences of the microorganisms included in the reference microbe portion 330.

The microbial source tracking system 322 can generate a microbial source log 332 that indicates one or more locations and one or more geological sources that correspond to microorganisms included in the monitoring microbial communities 326. Additionally, the microbial source log 332 can indicate one or more times that the microorganisms become present in the monitoring microbial communities 326. For example, monitoring samples from the monitoring wellbores can be collected at a number of times after injection of the one or more carbon-based gases into the injection wellbore. In one or more illustrative examples, a time interval between the collection of monitoring samples can be no greater than 4 hours, no greater than 6 hours, no greater than 12 hours, no greater than 18 hours, no greater than 24 hours, no greater than 48 hours, no greater than 7 days, no greater than 14 days, no greater than one month, no greater than 3 months, no greater than 6 months, or no greater than 12 months. As monitoring samples are collected over a period of time, the contribution of the reference microbe portion 330 to the monitoring microbial communities 326 can increase as more fluid from the one or more subsurface geological features at the location of the carbon-based gas injection site is displaced by the injection of the carbon-based gases and moves toward the one or more monitoring wellbores. In one or more further examples, the microbial source log 332 can indicate an abundance of microorganisms included in the monitoring microbial communities 326 at one or more periods of time.

In various examples, the microbial source log 332 can indicate individual microorganisms present in the monitoring microbial communities 326 and at least one of a subsurface geological feature that is a source of the individual microorganisms, a location of the subsurface geological features (e.g., injection site or monitoring wellbore), an abundance of the microorganisms present in the monitoring microbial communities 326, or changes in the composition and/or abundance of microorganisms included in the monitoring microbial communities 326 over a period of time. In one or more illustrative examples, the microbial source log 332 can also indicate a probability of a given subsurface geological feature being a source of microorganisms present in the monitoring microbial communities.

The tracking analysis system 320 can include a gas location identification system 334. The gas location identification system 334 can analyze the microbial source log 332 to determine gas location data 336. The gas location data 336 can indicate estimates of locations of one or more carbon-based gases injected into one or more subsurface geological features. In one or more examples, the gas location identification system 334 can determine that as an abundance of one or more microorganisms included in the reference microbe portion 330 of the monitoring microbial communities 326 increases, the one or more carbon-based gases are displacing an increasing amount of fluid in the one or more geological features that are the source of the one or more microorganisms with increasing abundance. In these scenarios, the gas location identification system 334 can determine a distance that the one or more carbon-based gases have traveled within a subsurface geological feature from the injection site of the one or more carbon-based gases to the one or more monitoring wellbores based on the increase in abundance of the microorganisms present in the reference microbe portion 330.

In one or more examples, the gas location identification system 334 can also determine a dissolution front of a plume of the one or more carbon-based gases moving through one or more subsurface geological features. For example, as the one or more carbon-based gases move into an area a change in pH can occur in locations where the one or more carbon-bases are present. In one or more examples, an environment in which the one or more carbon-based gases are present can become more acidic, thus lowering the pH of the environment. The lower pH in the regions of the subsurface geological features where the one or more carbon-based gases are present can impact the microbial communities located in these regions. In at least some examples, at least a portion of the microorganisms present in a region of a subsurface geological feature can die when the pH of the region is lowered due to the presence of the one or more carbon-based gases. In one or more illustrative examples, the amount of proteobacteria present in a location where the one or more carbon-based gases are present can decrease while and the retention of Sulphate Reducing Bacteria (SRB) and halobacterium can take place. In various examples, the microbial source log 332 can indicate when the one or more carbon-based gases have reached a monitoring wellbore by showing the changes in microbial communities that take place when the pH is lowered in the regions where the one or more carbon-based gases are present. In at least some examples, a period of time after the dissolution front caused by the presence of the one or more carbon-based gases has passed, the abundance of microorganisms that was reduced when the carbon-based gas plume front first passed through the region can begin to increase as an amount of repopulation of at least a portion of the microorganisms takes place in the region where the dissolution front has passed.

In various examples, the gas location identification system 334 can cause one or more user interfaces to be generated indicating locations of the one or more carbon-based gases within one or more subsurface geological formations. For example, the gas location identification system 334 can generate one or more models that indicate the presence of the one or more carbon-based gases within one or more subsurface geological formations. Additionally, the gas location identification system 314 can cause one or more user interfaces to be generated showing estimates of where the locations of the one or more carbon-based gases will move at one or more future time periods. To illustrate, the gas location identification system 334 can generate one or more models based on the microbial source log 332 that predict movement of the one or more carbon-based gases within one or more subsurface geological features.

Additionally, the tracking and analysis system 320 can include a gas location analysis system 338. The gas location analysis system 338 can analyze the gas location data 336 for a number of different purposes 340. For example, the gas location analysis system 338 can analyze the gas location data 336 to monitor for contamination of freshwater aquifers by the one or more carbon-based gases injected into the injection wellbore. To illustrate, the gas location analysis system 338 can determine when a location of the one or more carbon-based gases moves out of a rock-based subsurface geological feature into a freshwater aquifer. Additionally, the gas location analysis system 338 can analyze the gas location data 336 to determine when the one or more carbon-based gases move through a breach in caprock. In one or more illustrative examples, the gas location analysis system 338 can analyze the gas location data 336 to determine when one or more carbon-based gases have moved from a subsurface geological feature below the caprock to an additional subsurface geological feature above the caprock.

In one or more further examples, the gas location analysis system 338 can analyze the gas location data 336 in conjunction with other data gathered from subsurface geological features. In one or more illustrative examples, the gas location analysis system 338 can analyze the gas location data 336 in conjunction with pressure data collected from one or more subsurface geological features. In this way, the gas location analysis system 338 can determine a pressure profile within one or more subsurface geological features. In various examples, the pressure profile can indicate portions of one or more subsurface geological features in which an amount of pressure can impact the integrity of the subsurface geological features, such as caprock integrity. In at least some examples where the gas location analysis system 338 determines that the pressure in one or more regions of a subsurface geological feature exceeds a threshold pressure, one or more pressure mitigation strategies can be employed to reduce the pressure or minimize the pressure build up in the one or more regions.

In one or more additional examples, the gas location analysis system 338 can analyze the gas location data 336 to determine a capacity of one or more geological features to store one or more carbon-based gases. In at least some examples, the gas location analysis system 338 can analyze the gas location data 336 to determine a remaining capacity of one or more geological features to store one or more carbon-based gases. In one or more illustrative examples, the gas location analysis system 338 can analyze the gas location data 336 in conjunction with at least one of pressure data or temperature data collected from one or more subsurface geological features to determine a remaining capacity of the one or more subsurface geological features to store one or more carbon-based gases.

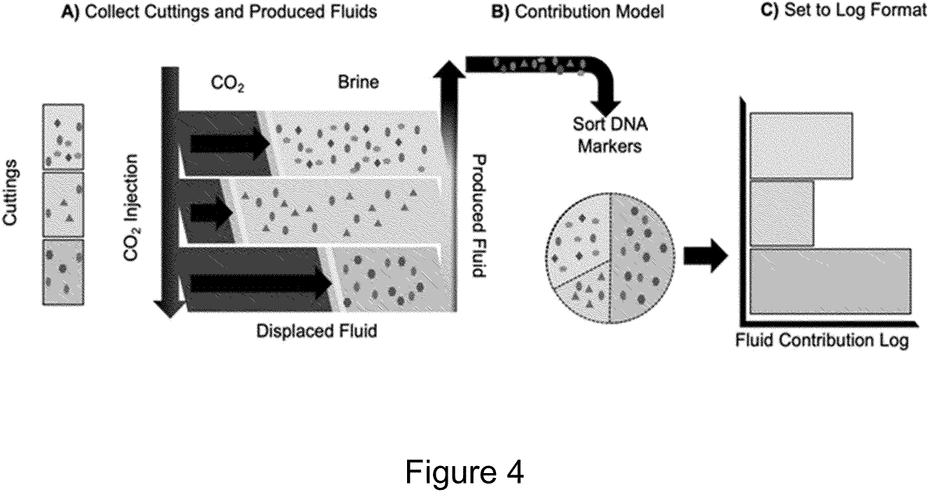

FIG. 4 is a diagram showing that microbial compositions of fluid located in subsurface geological features change as the fluid is displaced by injected carbon dioxide, according to one or more examples. For example, first microbial communities located in subsurface geological features can be determined based on genetic material extracted from cuttings obtained from a CO₂ injection site. As CO₂ is injected into the subsurface geological features, fluid present in the subsurface geological features is displaced and moves toward a monitoring wellbore or a produced fluids extraction wellbore. This can cause second microbial communities at the location of the monitoring wellbore or the produced fluids extraction wellbore to change. To illustrate, the first microbial communities originating from subsurface geological features present at the CO₂ inj ection site can become mixed with the second microbial communities present at the location of the monitoring wellbore or the produced fluids extraction wellbore in response to the CO₂ displacing fluid in the subsurface geological features at the CO₂ injection site to produce a mixture of microbial communities including at least a portion of the first microbial communities and the second microbial communities.

Computational techniques can be implemented to determine an amount of contribution to the mixture of microbial communities made by at least a portion of the first microbial communities. To illustrate, the computational techniques can separate the amount of contribution to the mixture of microbial communities made by the first microbial communities originating at the location of the CO₂ injection site. In various examples, the computational techniques can determine respective sources for the microorganisms present in the second microbial communities where the sources correspond to subsurface geological features located at the CO₂ injection and/or subsurface geological features located at the location of the monitoring wellbore or the produced fluids extraction wellbore. The amount of contribution of individual subsurface geological features can be used to determine a fluid contribution log indicating the respective amounts of contribution made by the individual subsurface geological features to the mixture of microbial communities present at the location of the monitoring wellbore or the produced fluids extraction wellbore.

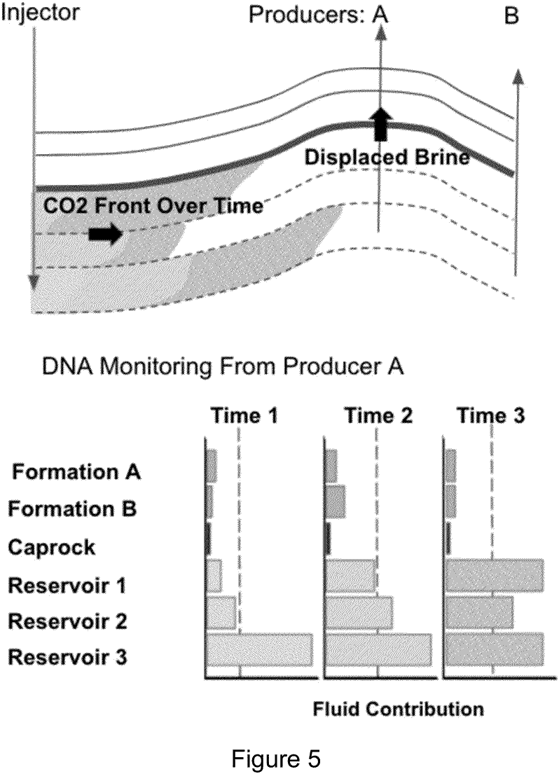

FIG. 5 is a diagram illustrating the movement of carbon dioxide from a number of geological features over time determined based on monitoring changes in genetic material extracted from samples collected at a number of locations, according to one or more examples. For example, FIG. 5 indicates movement of displaced brine between a CO₂ injection site and a first producer wellbore labeled “A” and a second producer wellbore labeled “B”. The movement of the displaced brine is shown by the contribution of microbial communities present in individual subsurface geological formations to a mixture of microbial communities present at the first produce wellbore and the second producer wellbore at different times, Time 1, Time 2, and Time 3. The contribution of individual microbial communities to the mixture of microbial communities present at the first produce wellbore and the second producer wellbore can be determined based on an amount of fluid displaced in the individual subsurface geological features by the injection of the CO₂. In one or more examples, the amount of fluid displaced within the individual subsurface geological features in response to the injection of CO₂ can be determined based on changes to the composition of the microbial communities present in subsurface geological features located at the first producer wellbore and/or the second produce wellbore.

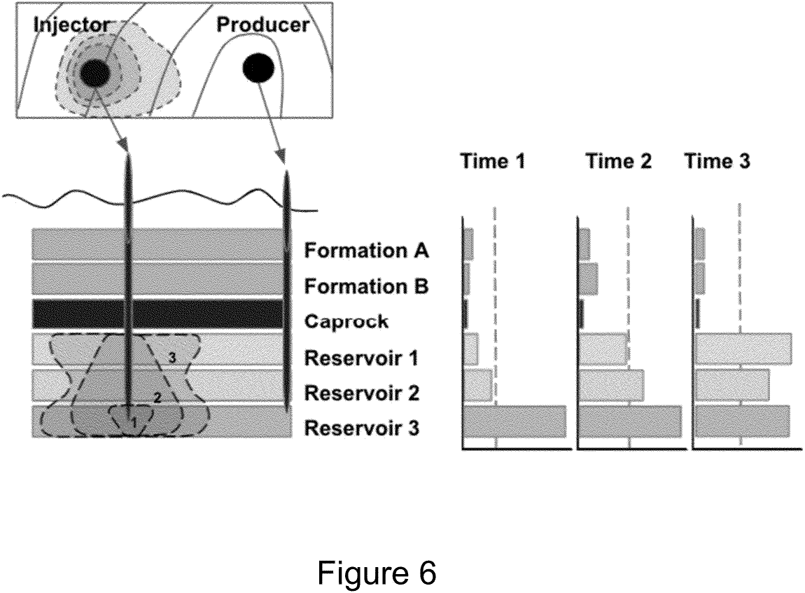

FIG. 6 is a diagram illustrating the flow of carbon dioxide in a number of geological features based on analyzing genetic material of microbial communities disposed in fluids present in the number of geological features, according to one or more examples. FIG. 6 illustrates the movement of carbon-based gas through individual subsurface geological features based on an amount of contribution of microbial communities present in the subsurface geological features at the carbon-based gas injection site to the microbial communities present in the subsurface geological features at the location of a producer wellbore at a number of times. FIG. 6 shows that the amount of movement of the carbon-based gas within the subsurface geological features corresponds to the amount of contribution of the microbial communities located in the individual subsurface geological features at the carbon-based gas injection point to the microbial communities located in the individual subsurface geological features at the producer wellbore location.

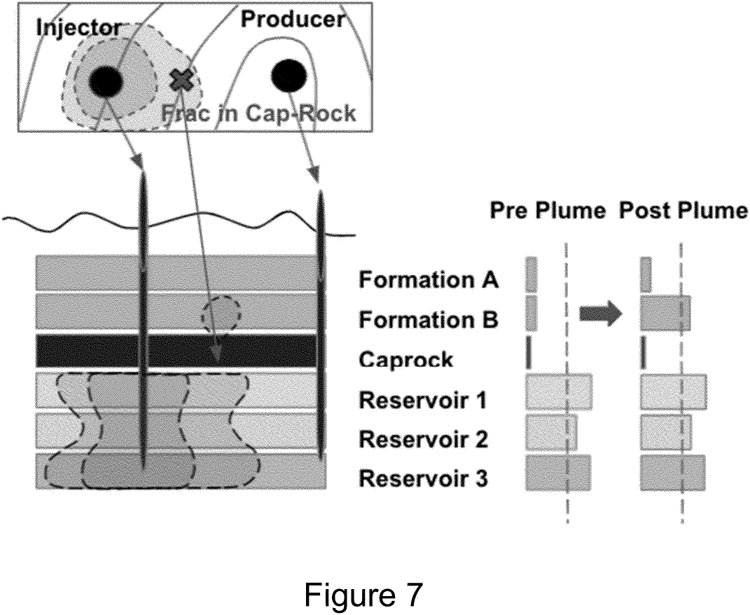

FIG. 7 is a diagram illustrating the detection of a fracture in caprock based on analyzing genetic material of microbial communities disposed in fluids present in the number of geological features, according to one or more examples. In the illustrative example of FIG. 7, a fracture in caprock is determined based on a contribution to the mixture of microbial communities at the producer wellbore location by microorganisms present in a rock formation above the caprock layer. The microbial community present in the rock formation above the caprock layer can be distinguished from the microbial community present in the reservoir below the caprock layer based on the presence of different microorganisms in the microbial community of the rock formation above the caprock layer with respect to the microorganisms present in the reservoir below the caprock layer.

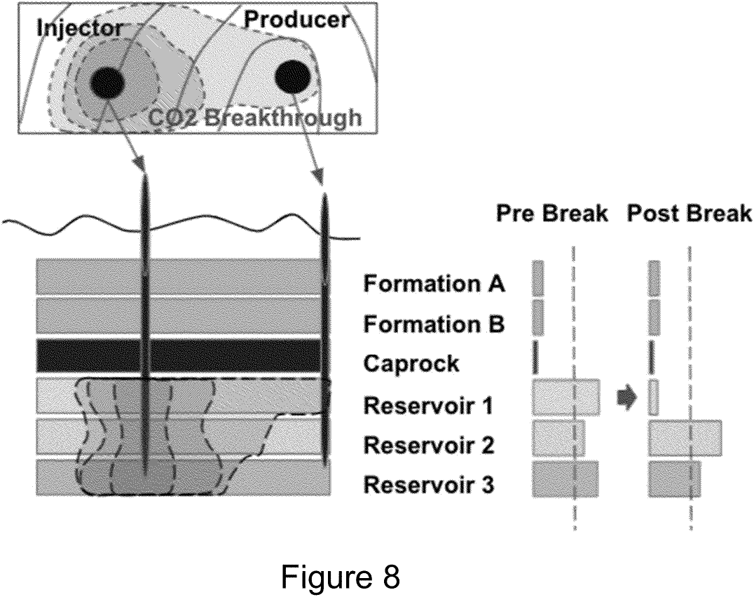

FIG. 8 is a diagram illustrating the detection of carbon dioxide at a monitoring borehole or a producer borehole based on analyzing genetic material of microbial communities disposed in fluids present in the number of geological features, according to one or more examples. In the illustrative example of FIG. 8, carbon-based gas present in a first reservoir has displaced fluid in the first reservoir to the point where the carbon-based gas has reached the location of a producer wellbore. In this scenario, the microbial community present in the first reservoir at the location of the producer wellbore is changed by the presence of the carbon-based gas. For example, the carbon-based gas can cause a pH at the location of the producer wellbore in the first reservoir to decrease to levels where the microorganisms previously present in the first reservoir at the location of the producer wellbore are unable to survive. Thus, the decrease in microorganisms present in the first reservoir at the location of the producer wellbore indicates a breakthrough of CO₂ at the location of the producer wellbore in the first reservoir.

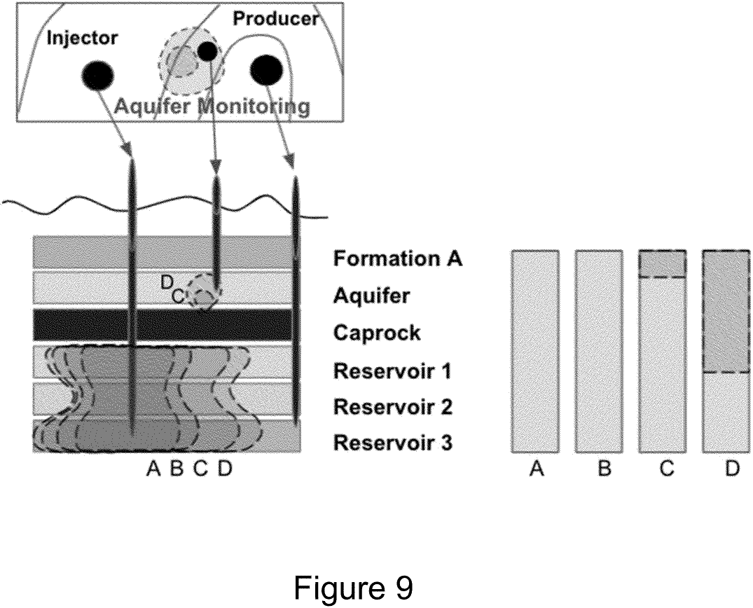

FIG. 9 is a diagram illustrating the detection of the presence of carbon dioxide in an aquifer based on analyzing genetic material of microbial communities disposed in fluids present in the number of geological features, according to one or more examples. In the illustrative example of FIG. 9, the amount of carbon-based gas present in the aquifer is increasing over time. For example, the amount of carbon-based gas moving into the aquifer from the first reservoir through a fracture in the caprock layer is increasing as more carbon-based gas moves into the first reservoir. The presence of the carbon-based gas in the reservoir can be determined by collecting samples from the aquifer from a monitoring wellbore that is located between a carbon-based gas injection site and a producer wellbore. The genetic material extracted from samples obtained from the monitoring wellbore can be analyzed to determine a microbial community present in the aquifer. As the carbon-based gas moves into the aquifer from the first reservoir through one or more fractures in the caprock layer, an abundance of at least a portion of the microorganisms present in the aquifer at the location of the monitoring wellbore may decrease due to changes in pH caused by the present of the carbon-based gas in the aquifer at the location of the monitoring wellbore.

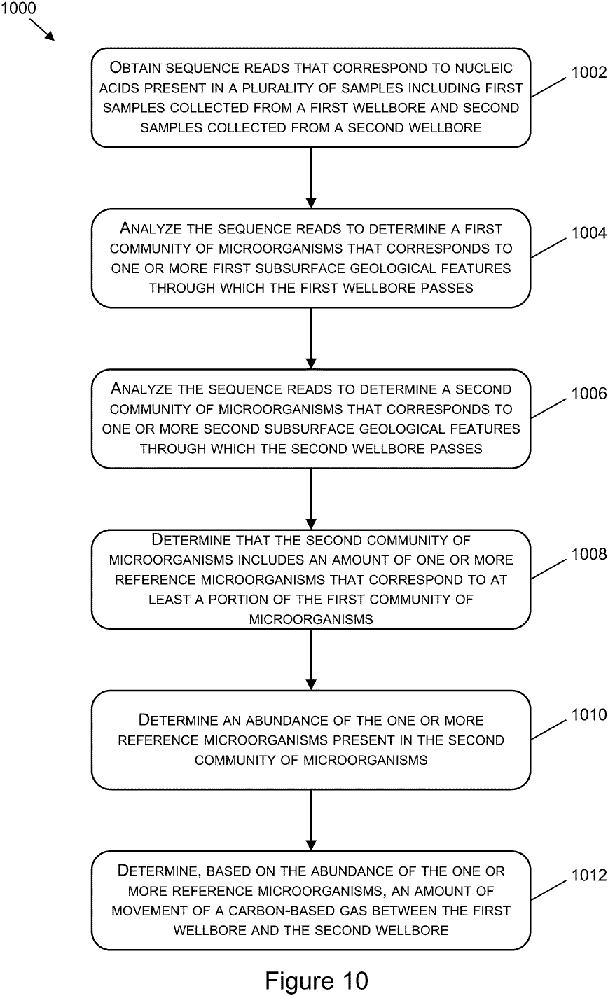

FIG. 10 is a flow diagram of an example process to determine the flow of carbon dioxide through one or more subsurface geological features, according to one or more examples. At operation 1002, the process 1000 includes obtaining sequence reads that correspond to nucleic acids present n a plurality of samples. The plurality of samples can include first samples collected from a first wellbore and second samples collected from a second wellbore.

The process 1000 can also include, at operation 1004, analyzing the sequence reads to determine a first community of microorganisms that corresponds to one or more first subsurface geological features through which the first wellbore passes. Additionally, at operation 1006, the process 1000 can include analyzing the sequence reads to determine a second community of microorganism that corresponds to one or more second subsurface geological features through which the second wellbore passes.

Further, the process 1000 can include, at operation 1008, determining that the second community of microorganisms includes an amount of one or more reference microorganisms. The one or more reference microorganisms can correspond to at least a portion of the first community of microorganisms. At operation 1010, the process 1000 can include determining an abundance of the one or more reference organisms present in the second community of microorganisms.

In at least some examples, one or more machine learning techniques and/or statistical techniques can be implemented to determine sources of microbial communities and to distinguish between the microbial communities. Additionally, one or more machine learning techniques and/or statistical techniques can be implemented to determine probabilities that a given source is attributed to a microbial community. For example, one or more machine learning approaches can be used to determine probabilities of sources of microbial communities, such as one or more support vector machines or one or more k-nearest neighbors algorithms. Additionally, at least one of Unifrac distance or principle component analysis can be used to distinguish between microbial communities.

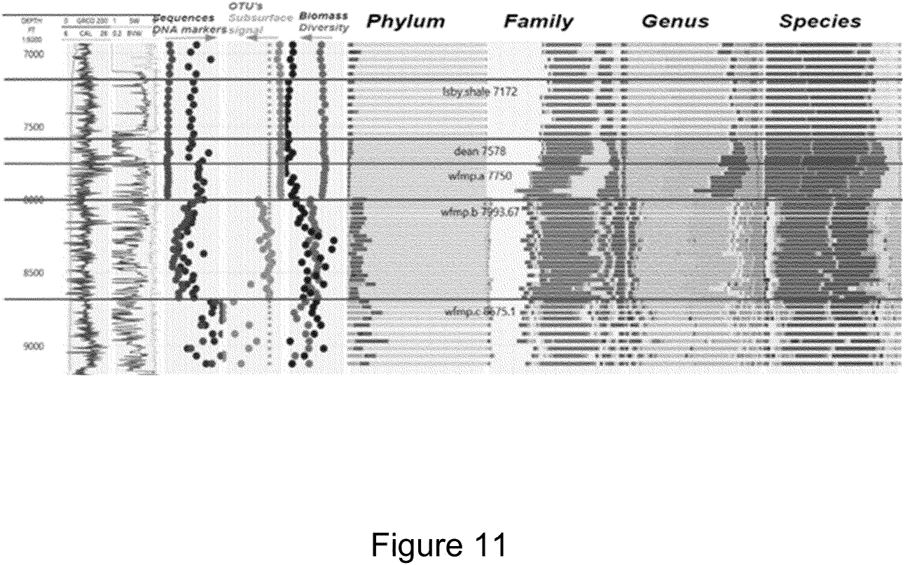

FIG. 11 is a chart showing the log of microbial communities located at a number of depths of one or more subsurface geological features. The microorganisms represented in FIG. 11 can correspond to a number of operational taxonomic units. Additionally, an abundance of phyla, families, genera, and species represented by the microbial communities is also represented in the illustrative example of FIG. 11 as well as logs calculated from the sequence data, such as diversity and biomass. The illustrative example of FIG. 11 corresponds to a DNA stratigraphy log measured from cuttings obtained from a wellbore, where the DNA stratigraphy log is integrated with petrophysical logs and formation top.

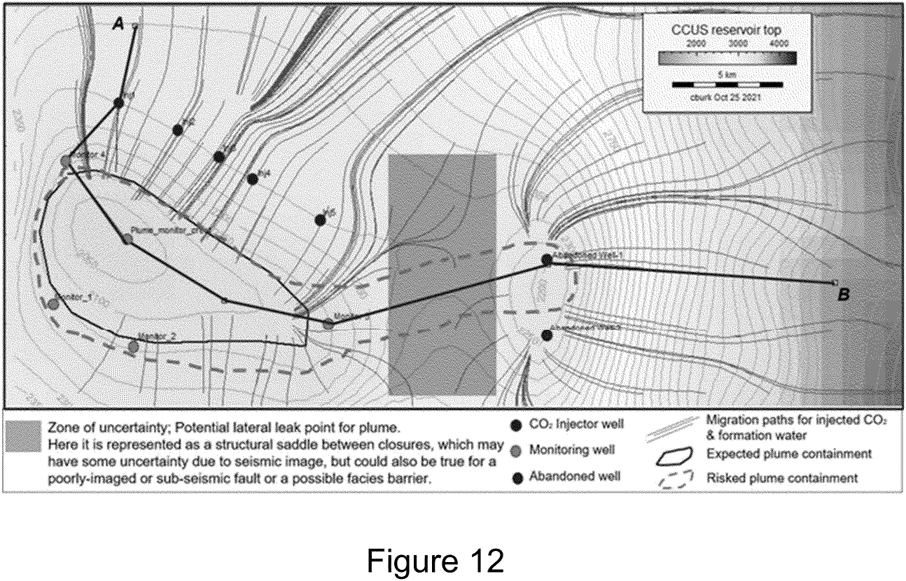

FIG. 12 is a diagram of a subsurface model indicating the flow of carbon dioxide through one or more subsurface geological features. The illustrative example of FIG. 12 represents a subsurface model indicating movement of carbon dioxide through a homogenous sandstone reservoir and a proposed complex for the storage of carbon dioxide.

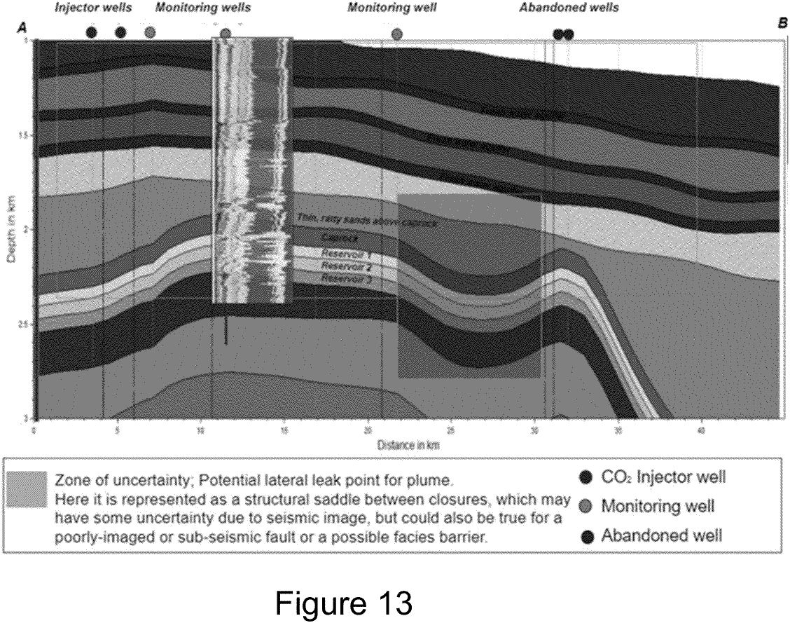

FIG. 13 is a diagram showing a cross-section of a model of a number of geological features indicating microbial communities present in the number of subsurface geological features. The cross-section of FIG. 11 corresponds to the lines of section indicated in FIG. 12.

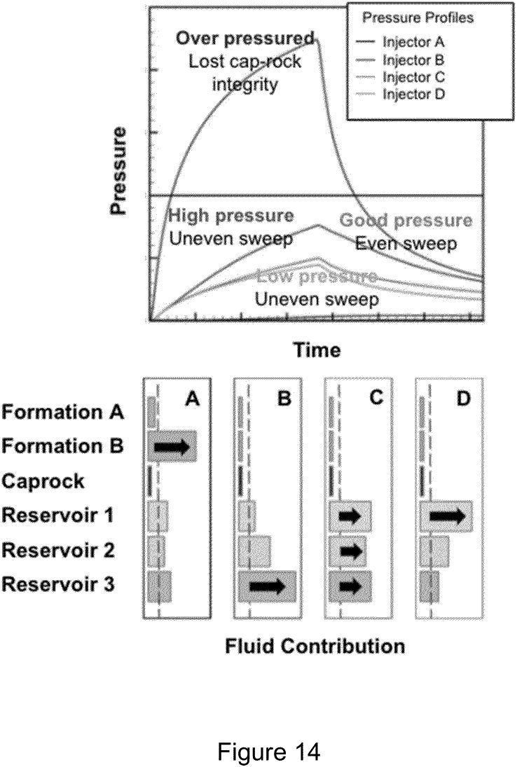

FIG. 14 is a diagram showing the impact on the movement of carbon dioxide through a number of subsurface geological features based on different carbon dioxide injection pressures. The modeling of the fluid contribution of a number of subsurface geological features under different pressure profiles can identify conditions for the injection of carbon-based gas into subsurface geological features and instances where pressure mitigation strategies may be employed.

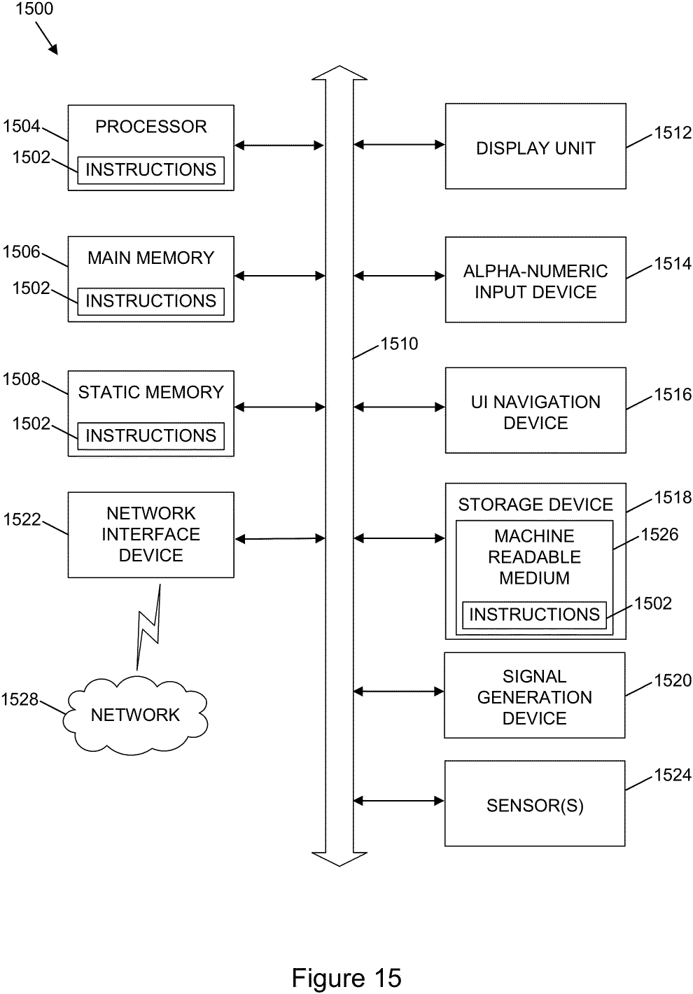

FIG. 15 illustrates a diagrammatic representation of a machine in the form of a computer system within which a set of instructions may be executed for causing the machine to perform any one or more of the methodologies discussed herein, according to one or more examples. Specifically, FIG. 15 shows a diagrammatic representation of the machine 1500 in the example form of a computer system, within which instructions 1502 (e.g., software, a program, an application, an applet, an app, or other executable code) for causing the machine 1500 to perform any one or more of the methodologies discussed herein may be executed.

The instructions 1502 transform the general, non-programmed machine 1500 into a particular machine 1500 programmed to carry out the described and illustrated functions in the manner described. In alternative implementations, the machine 1500 operates as a standalone device or may be coupled (e.g., networked) to other machines. In a networked deployment, the machine 1500 may operate in the capacity of a server machine or a client machine in a server-client network environment, or as a peer machine in a peer-to-peer (or distributed) network environment. The machine 1500 may comprise, but not be limited to, a server computer, a client computer, a personal computer (PC), a tablet computer, a laptop computer, a netbook, a set-top box (STB), a personal digital assistant (PDA), an entertainment media system, a cellular telephone, a smart phone, a mobile device, a wearable device (e.g., a smart watch), a smart home device (e.g., a smart appliance), other smart devices, a web appliance, a network router, a network switch, a network bridge, or any machine capable of executing the instructions 1502, sequentially or otherwise, that specify actions to be taken by the machine 1500. Further, while only a single machine 1500 is illustrated, the term “machine” shall also be taken to include a collection of machines 1500 that individually or jointly execute the instructions 1502 to perform any one or more of the methodologies discussed herein.

Examples of machine 1500 can include logic, one or more components, circuits (e.g., modules), or mechanisms. Circuits are tangible entities configured to perform certain operations. In an example, circuits can be arranged (e.g., internally or with respect to external entities such as other circuits) in a specified manner. In an example, one or more computer systems (e.g., a standalone, client or server computer system) or one or more hardware processors (processors) can be configured by software (e.g., instructions, an application portion, or an application) as a circuit that operates to perform certain operations as described herein. In an example, the software can reside (1) on a non-transitory machine readable medium or (2) in a transmission signal. In an example, the software, when executed by the underlying hardware of the circuit, causes the circuit to perform the certain operations.

In an example, a circuit can be implemented mechanically or electronically. For example, a circuit can comprise dedicated circuitry or logic that is specifically configured to perform one or more techniques such as discussed above, such as including a special-purpose processor, a field programmable gate array (FPGA) or an application-specific integrated circuit (ASIC). In an example, a circuit can comprise programmable logic (e.g., circuitry, as encompassed within a general-purpose processor or other programmable processor) that can be temporarily configured (e.g., by software) to perform the certain operations. It will be appreciated that the decision to implement a circuit mechanically (e.g., in dedicated and permanently configured circuitry), or in temporarily configured circuitry (e.g., configured by software) can be driven by cost and time considerations.

Accordingly, the term “circuit” is understood to encompass a tangible entity, be that an entity that is physically constructed, permanently configured (e.g., hardwired), or temporarily (e.g., transitorily) configured (e.g., programmed) to operate in a specified manner or to perform specified operations. In an example, given a plurality of temporarily configured circuits, each of the circuits need not be configured or instantiated at any one instance in time. For example, where the circuits comprise a general-purpose processor configured via software, the general-purpose processor can be configured as respective different circuits at different times. Software can accordingly configure a processor, for example, to constitute a particular circuit at one instance of time and to constitute a different circuit at a different instance of time.

In an example, circuits can provide information to, and receive information from, other circuits. In this example, the circuits can be regarded as being communicatively coupled to one or more other circuits. Where multiple of such circuits exist contemporaneously, communications can be achieved through signal transmission (e.g., over appropriate circuits and buses) that connect the circuits. In implementations in which multiple circuits are configured or instantiated at different times, communications between such circuits can be achieved, for example, through the storage and retrieval of information in memory structures to which the multiple circuits have access. For example, one circuit can perform an operation and store the output of that operation in a memory device to which it is communicatively coupled. A further circuit can then, at a later time, access the memory device to retrieve and process the stored output. In an example, circuits can be configured to initiate or receive communications with input or output devices and can operate on a resource (e.g., a collection of information).

The various operations of method examples described herein can be performed, at least partially, by one or more processors that are temporarily configured (e.g., by software) or permanently configured to perform the relevant operations. Whether temporarily or permanently configured, such processors can constitute processor-implemented circuits that operate to perform one or more operations or functions. In an example, the circuits referred to herein can comprise processor-implemented circuits.

Similarly, the methods described herein can be at least partially processor implemented. For example, at least some of the operations of a method can be performed by one or processors or processor-implemented circuits. The performance of certain of the operations can be distributed among the one or more processors, not only residing within a single machine, but deployed across a number of machines. In an example, the processor or processors can be located in a single location (e.g., within a home environment, an office environment or as a server farm), while in other examples the processors can be distributed across a number of locations.

The one or more processors can also operate to support performance of the relevant operations in a “cloud computing” environment or as a “software as a service”

(SaaS). For example, at least some of the operations can be performed by a group of computers (as examples of machines including processors), with these operations being accessible via a network (e.g., the Internet) and via one or more appropriate interfaces (e.g., Application Program Interfaces (APIs).)

Example implementations (e.g., apparatus, systems, or methods) can be implemented in digital electronic circuitry, in computer hardware, in firmware, in software, or in any combination thereof. Example implementations can be implemented using a computer program product (e.g., a computer program, tangibly embodied in an information carrier or in a machine readable medium, for execution by, or to control the operation of, data processing apparatus such as a programmable processor, a computer, or multiple computers).

A computer program can be written in any form of programming language, including compiled or interpreted languages, and it can be deployed in any form, including as a stand-alone program or as a software module, subroutine, or other unit suitable for use in a computing environment. A computer program can be deployed to be executed on one computer or on multiple computers at one site or distributed across multiple sites and interconnected by a communication network.

In an example, operations can be performed by one or more programmable processors executing a computer program to perform functions by operating on input data and generating output. Examples of method operations can also be performed by, and example apparatus can be implemented as, special purpose logic circuitry (e.g., a field programmable gate array (FPGA) or an application-specific integrated circuit (ASIC)).

The computing system can include clients and servers. A client and server are generally remote from each other and generally interact through a communication network. The relationship of client and server arises by virtue of computer programs running on the respective computers and having a client-server relationship to each other. In implementations deploying a programmable computing system, it will be appreciated that both hardware and software architectures require consideration. Specifically, it will be appreciated that the choice of whether to implement certain functionality in permanently configured hardware (e.g., an ASIC), in temporarily configured hardware (e.g., a combination of software and a programmable processor), or a combination of permanently and temporarily configured hardware can be a design choice. Below are set out hardware (e.g., machine 1500) and software architectures that can be deployed in example implementations.

In an example, the machine 1500 can operate as a standalone device or the machine 1500 can be connected (e.g., networked) to other machines.

In a networked deployment, the machine 1500 can operate in the capacity of either a server or a client machine in server-client network environments. In an example, machine 1500 can act as a peer machine in peer-to-peer (or other distributed) network environments. The machine 1500 can be a personal computer (PC), a tablet PC, a set-top box (STB), a Personal Digital Assistant (PDA), a mobile telephone, a web appliance, a network router, switch or bridge, or any machine capable of executing instructions (sequential or otherwise) specifying actions to be taken (e.g., performed) by the machine 1500. Further, while only a single machine 1500 is illustrated, the term “computing device” shall also be taken to include any collection of machines that individually or jointly execute a set (or multiple sets) of instructions to perform any one or more of the methodologies discussed herein.

Example machine 1500 can include a processor 1504 (e.g., a central processing unit CPU), a graphics processing unit (GPU) or both), a main memory 1506 and a static memory 1508, some or all of which can communicate with each other via a bus 1510. The machine 1500 can further include a display unit 1512, an alphanumeric input device 1514 (e.g., a keyboard), and a user interface (UI) navigation device 1516 (e.g., a mouse). In an example, the display unit 1512, input device 1514 and UI navigation device 1516 can be a touch screen display. The machine 1500 can additionally include a storage device (e.g., drive unit) 1518, a signal generation device 1520 (e.g., a speaker), a network interface device 1522, and one or more sensors 1524, such as a global positioning system (GPS) sensor, compass, accelerometer, or another sensor.

The storage device 1518 can include a machine readable medium 1526 on which is stored one or more sets of data structures or instructions 1502 (e.g., software) embodying or utilized by any one or more of the methodologies or functions described herein. The instructions 1502 can also reside, completely or at least partially, within the main memory 1506, within static memory 1508, or within the processor 1504 during execution thereof by the machine 1500. In an example, one or any combination of the processor 1504, the main memory 1506, the static memory 1508, or the storage device 1518 can constitute machine readable media.

While the machine readable medium 1526 is illustrated as a single medium, the term “machine readable medium” can include a single medium or multiple media (e.g., a centralized or distributed database, and/or associated caches and servers) that configured to store the one or more instructions 1502. The term “machine readable medium” can also be taken to include any tangible medium that is capable of storing, encoding, or carrying instructions for execution by the machine and that cause the machine to perform any one or more of the methodologies of the present disclosure or that is capable of storing, encoding or carrying data structures utilized by or associated with such instructions. The term “machine readable medium” can accordingly be taken to include, but not be limited to, solid-state memories, and optical and magnetic media. Specific examples of machine-readable media can include non-volatile memory, including, by way of example, semiconductor memory devices (e.g., Electrically Programmable Read-Only Memory

(EPROM), Electrically Erasable Programmable Read-Only Memory (EEPROM)) and flash memory devices; magnetic disks such as internal hard disks and removable disks; magneto-optical disks; and CD-ROM and DVD-ROM disks.

The instructions 1502 can further be transmitted or received over a communications network 1528 using a transmission medium via the network interface device 1522 utilizing any one of a number of transfer protocols (e.g., frame relay, IP, TCP, UDP, HTTP, etc.). Example communication networks can include a local area network (LAN), a wide area network (WAN), a packet data network (e.g., the Internet), mobile telephone networks (e.g., cellular networks), Plain Old Telephone (POTS) networks, and wireless data networks (e.g., IEEE 1502.11 standards family known as Wi-Fi®, IEEE 1502.16 standards family known as WiMax®), peer-to-peer (P2P) networks, among others. The term “transmission medium” shall be taken to include any intangible medium that is capable of storing, encoding or carrying instructions for execution by the machine, and includes digital or analog communications signals or other intangible medium to facilitate communication of such software.

What is claimed is:

1. A method comprising:
obtaining, by a computing system including one or more processors and memory, sequence reads that correspond to nucleic acids present in a plurality of samples, the plurality of samples including first samples collected from a first wellbore and second samples collected from a second wellbore;
analyzing, by the computing system, the sequence reads to determine a first community of microorganisms that corresponds to one or more first subsurface geological features through which the first wellbore passes;
analyzing, by the computing system, the sequence reads to determine a second community of microorganisms that corresponds to one or more second subsurface geological features through which the second wellbore passes;
determining, by the computing system, that the second community of microorganisms includes an amount of one or more reference microorganism that correspond to at least a portion of the microorganisms of the first community of microorganisms;
determining, by the computing system, an abundance of the one or more reference microorganisms present in the second community of microorganisms; and
determining, by the computing system and based on the abundance of the one or more reference microorganisms, an amount of movement of a carbon-based gas between the first wellbore and the second wellbore.

2. The method of claim 1, wherein:
an amount of the carbon-based gas is injected into the first wellbore;
the first samples are collected before the amount of carbon-based gas is injected into the first wellbore; and
the second samples are collected after the amount of carbon-based gas is injected into the first wellbore.

3. The method of claim 1, wherein:
the first samples are collected from a plurality of depths below a surface; and
individual depths of the plurality of depths correspond to individual subsurface geological features.

4. The method of claim 3, wherein the individual subsurface geological features include at least one of a freshwater aquifer, a caprock layer, a rock formation, or a brine reservoir.

5. The method of claim 3, comprising:
analyzing, by the computing system, a first portion of the sequence reads that corresponds to one or more samples collected from a first individual subsurface geological feature to determine a first reference community of microorganisms that corresponds to a first individual subsurface geological feature; and
analyzing, by the computing system, a second portion of the sequence reads that corresponds to one or more additional samples collected from a second individual subsurface geological feature to determine a second reference community of microorganisms that corresponds to a second individual subsurface geological feature.

6. The method of claim 5, comprising:
analyzing, by the computing system, a first number of the sequence reads that corresponds to first genomic sequences of the first reference community of microorganisms present in the second community of microorganisms;
determining, by the computing system and based on the first number of the sequence reads, a first abundance of the first reference community of microorganisms present in the second community of microorganisms;
analyzing, by the computing system, a second number of the sequence reads that corresponds to second genomic sequences of the second reference community of microorganisms present in the second community of microorganisms; and
determining, by the computing system and based on the second number of the sequence reads, a second abundance of the second reference community of microorganisms present in the second community of microorganisms.

7. The method of claim 6, comprising:
determining, by the computing system, a microbial source log indicating:
a first amount of the second community of microorganisms that corresponds to the first individual subsurface geological feature based on the first abundance of the first reference community of microorganisms present in the second community of microorganisms; and
a second amount of the second community of microorganisms that corresponds to the second individual subsurface geological feature based on the second abundance of the second reference community of microorganisms present in the second community of microorganism.

8. The method of claim 6, wherein:
the first abundance of the first reference community of microorganisms present in the second community of microorganisms corresponds to a first amount of fluid displaced in the first individual subsurface geological feature in response to injection of one or more carbon-based gases at the first wellbore; and
the second abundance of the second reference community of microorganisms present in the second community of microorganisms corresponds to a second amount of fluid displaced in the second individual subsurface geological features in response to the injection of the one or more carbon-based gases at the first wellbore.

9. The method of claim 8, comprising:
determining, by the computing system, a first location of the one or more carbon-based gases in the first individual subsurface geological feature based on the first amount of fluid displaced in the first individual subsurface geological feature in response to injection of one or more carbon-based gases at the first wellbore; and
determining, by the computing system, a second location of the one or more carbon-based gases in the second individual subsurface geological features based on the second amount fluid displaced in the second individual subsurface geological features in response to the injection of the one or more carbon-based gases at the first wellbore.

10. The method of claim 9, wherein the second individual subsurface geological feature is a freshwater aquifer; and the method comprising:
determining, by the computing system, that an amount of the one or more carbon-based gases is present in the freshwater aquifer based on the second location of the one or more carbon-based gases.

11. The method of claim 9, wherein the first individual subsurface geological feature is located above a caprock layer and the second individual subsurface geological feature is located below a caprock layer, and the method comprising:
determining, by the computing system, that a breach of the caprock layer has occurred based on the first location of the one or more carbon-based gases and the second location of the one or more carbon-based gases.

12. The method of claim 9, comprising:
obtaining, by the computing system, pressure measurements from one or more pressure sensors present in at least one of the first individual subsurface geological feature or the second individual subsurface geological feature;
analyzing, by the computing system, the pressure measurements in conjunction with the first location of the carbon-based gases and the second location of the carbon-based gases to determine that a pressure threshold in at least one of the first individual subsurface geological feature or the second individual subsurface geological feature has been exceeded; and
determining one or more pressure mitigation operations to apply with respect to at least one of the first individual subsurface geological feature or the second individual subsurface geological feature.

13. The method of claim 9, comprising:
analyzing, by the computing system, the first location of the one or more carbon-based gases and the second location of the one or more carbon-based gases to determine a storage capacity for the one or more carbon-based gases in at least one of the first individual subsurface geological feature or the second individual subsurface geological feature.

14. The method of claim 1, comprising:
extracting first genetic material from the first samples;
extracting second genetic material from the second samples; and
performing one or more amplification and sequencing processes with respect to the first genetic material and the second genetic material to generate the sequence reads.

15. The method of claim 1, comprising:
analyzing, by the computing system, the sequence reads with respect to genomic sequences of microorganisms to determine an amount of identity between individual sequence reads and the genomic sequences of the microorganisms; and
determining, by the computing system, than an individual sequence read corresponds to a genomic sequence of a microorganism based on the amount of identity between the individual sequence read and the genomic sequence of the microorganism being at least a threshold amount of identity.

16. The method of claim 1, comprising:
determining, by the computing system, taxonomic classifications for at least a portion of microorganisms included in the first community of microorganisms and the second community of microorganisms.

17. A computing system comprising:
one or more hardware processors; and
memory storing computer-readable instructions that, when executed by the one or more hardware processors, cause the one or more hardware processors to perform operations comprising:
obtaining sequence reads that correspond to nucleic acids present in a plurality of samples, the plurality of samples including first samples collected from a first wellbore and second samples collected from a second wellbore;
analyzing the sequence reads to determine a first community of microorganisms that corresponds to one or more first subsurface geological features through which the first wellbore passes;
analyzing the sequence reads to determine a second community of microorganisms that corresponds to one or more second subsurface geological features through which the second wellbore passes;
determining that the second community of microorganisms includes one or more reference microorganism that correspond to at least a portion of the microorganisms of the first community of microorganisms;
determining an abundance of the one or more reference microorganisms present in the second community of microorganisms; and
determining, based on the abundance of the one or more reference microorganisms, an amount of movement of a carbon-based gas between the first wellbore and the second wellbore.

18. The computing system of claim 17, wherein the memory stores additional computer-readable instructions that, when executed by the one or more hardware processors, cause the one or more hardware processors to perform additional operations comprising:
determining that an additional abundance of one or more classifications of microorganisms present in the second community of microorganisms has decreased, the one or more classifications of microorganisms including microorganisms that are unable to survive in environments where greater than a threshold amount of the carbon-based gas is present; and
determining that a dissolution front of the carbon-based gas is present at a location of the second wellbore.

19. The computing system of claim 17, wherein the memory stores additional computer-readable instructions that, when executed by the one or more hardware processors, cause the one or more hardware processors to perform additional operations comprising:
determining locations of the one or more carbon-based gases in at least one of the one or more first subsurface geological features or the one or more second subsurface geological features based on one or more microorganisms originating in the one or more first subsurface geological features being present in the one or more second subsurface geological features; and
generating a model to predict the amount of movement of the carbon-based gas between the first wellbore and the second wellbore based on the locations of the one or more carbon-based gases in at least one of the one or more first subsurface geological features or the one or more second subsurface geological features.

20. The computing system of claim 19, wherein the memory stores additional computer-readable instructions that, when executed by the one or more hardware processors, cause the one or more hardware processors to perform additional operations comprising:
causing a user interface to be generated that includes one or more graphics indicating the amount of movement of the carbon-based gas between the first wellbore and the second wellbore.
